(19) United States
(12) Patent Application Publication
(10) Pub. No.: US 2005/0101520 A1
(43) Pub. Date: May 12, 2005

(54) CTLA4 compositions in the treatment of autism

(76) Inventor: Harumi Jyonouchi, Glen Ridge, NJ (US)

Correspondence Address: FISH & RICHARDSON PC, 225 FRANKLIN ST, BOSTON, MA, 02110, US

(21) Appl. No.: 10/403,675

(22) Filed: Mar. 31, 2003

Related U.S. Application Data
(60) Provisional application No. 60/368,625, filed on Mar. 29, 2002.

(51) Int. Cl. A61K 31/00

(52) U.S. Cl. 514/1

(57) ABSTRACT

The invention features methods for treating an individual exhibiting one or more symptoms of autistic syndrome disorder (ASD) and/or dietary protein intolerance (DPI), in which the methods include administering an effective amount of a CTLA4 composition to the individual, wherein one or more symptoms of ASD and/or DPI are improved.

### CROSS-REFERENCE TO RELATED APPLICATION

This application claims priority from U.S. Provisional Application 60/368,625, filed on Mar. 29, 2002, the contents of which are incorporated herein by reference in their entirety.

### FIELD OF THE INVENTION

This invention relates to the use of CTLA4 compositions in the treatment of autism and dietary protein intolerance.

### BACKGROUND OF THE INVENTION

Autism spectrum disorder (ASD) is a complex developmental disorder diagnosed on the basis of subjective criteria and its etiology appears heterogeneous.

Autism spectrum disorder (otherwise known as autistic disorder or autism) is a severely debilitating developmental disorder characterized by a profound deficiency in verbal communication and normal socialization. Autism is typically diagnosed in children between the ages of two or three and six with a diagnosis usually being made based on behavioral symptoms using the guidelines in the Diagnostic and Statistical Manual of Mental Disorders (DSM)(American Psychiatric Association, Washington, D.C., pages 66-71, 1994). The fourth edition of these guidelines, DSM-IV, identifies autistic disorder as one of five separate disorders under the general category of Pervasive Developmental Disorders.

A child is diagnosed as having autistic disorder if the child fits into all of categories A, B, and C as follows (American Psychiatric Association, supra, pages 70-71):
A. A total of six (or more) items from (1), (2), and (3), with at least two from (1), and one each from (2) and (3):
   (1) qualitative impairment in social interaction, as manifested by at least two of the following: (a) marked impairment in the use of multiple nonverbal behaviors such as eye-to-eye gaze, facial expression, body postures, and gestures to regulate social interaction; (b) failure to develop peer relationships appropriate to developmental level; (c) a lack of spontaneous seeking to share enjoyment, interests, or achievements with other people (e.g., by a lack of showing, bringing, or pointing out objects of interest); and (d) lack of social or emotional reciprocity.
   (2) qualitative impairments in communication as manifested by at least one of the following: (a) delay in, or total lack of, the development of spoken language (not accompanied by an attempt to compensate through alternative modes of communication such as gesture or mime); (b) in individuals with adequate speech, marked impairment in the ability to initiate or sustain a conversation with others; (c) stereotyped and repetitive use of language or idiosyncratic language; and (d) lack of varied, spontaneous make-believe play or social imitative play appropriate to developmental level.
   (3) restricted repetitive and stereotyped patterns of behavior, interests, and activities, as manifested by at least one of the following: (a) encompassing preoccupation with one or more stereotyped and restricted patterns of interest that is abnormal either in intensity or focus; (b) apparently inflexible adherence to specific, nonfunctional routines or rituals; (c) stereotyped and repetitive motor mannerisms (e.g., hand or finger flapping or twisting, or complex whole-body movements); and (d) persistent preoccupation with parts of objects.
B. Delays or abnormal functioning in at least one of the following areas, with onset prior to age 3 years:
   (1) social interaction,
   (2) language as used in social communication, or
   (3) symbolic or imaginative play.
C. The disturbance is not better accounted for by Rett's Disorder or Childhood Disintegrative Disorder.
   Although useful, diagnoses based on behavioral symptoms are necessarily subjective.

Many ASD children with developmental regression suffer from various gastrointestinal (GI) symptoms including GI cramping, chronic diarrhea/constipation, gastroesophageal reflux (GER), and bloating. Controlling GI symptoms appear associated with clinical improvement; many ASD children show clinical improvement following implementation of a gluten-free, casein-free (gf/cf) diet. Most of ASD children who respond to a restricted diet do not reveal specific IgE against dietary proteins (DPs) or positive skin test (ST) reactivity, and rarely exhibit immediate or anaphylactic reactions following ingestion of DPs to which the child reacts (Jyonouchi et al., J. Neuroimmunol., 120:170-9, 2001).

### SUMMARY OF THE INVENTION

The invention is based, in part, on the discovery that administration of CTLA4 compositions, such as CTLA4-Ig, to patients with autism spectrum disorder (ASD), for example, patients with ASD that have dietary protein intolerance (DPI), can reduce excessive amounts of IFN-γ produced in these patients, thereby lessening symptoms of DPI and/or ASD.

In general, the invention features methods for treating individuals exhibiting one or more symptoms of ASD by administering an effective amount of a CTLA4 composition to the individual, wherein one or more symptoms of ASD are improved.

The invention also features methods for treating individuals exhibiting one or more symptoms of ASD and DPI, by administering an effective amount of a CTLA4 composition to the individual, wherein one or more symptoms of ASD or DPI are improved.

The invention further features methods for treating individuals exhibiting one or more symptoms of DPI by administering an effective amount of a CTLA4 composition to the individual, and wherein one or more of the symptoms of DPI is improved.

In any of these methods, the individual can exhibit an elevated level of a cytokine (e.g., an inflammatory cytokine, such as IFN-γ), in which the method includes administering an effective amount of a CTLA4 composition (e.g., sufficient to decrease levels of IFN-γ in the blood of the individual) to the individual, wherein one or more symptoms of ASD are improved.

In addition, the individuals may exhibit one or more gastrointestinal symptoms, wherein administration of an effective amount of a CTLA4 composition to the individual results in one or more gastrointestinal symptoms being improved.

In the methods described herein, the effective amount of CTLA4 in the CTLA4 composition can range from about 0.1 mg/kg to about 10 mg/kg per administration (e.g., 0.5, 1.5, 5.0, or 10.0 mg/kg), the CTLA4 composition can be administered orally or transdermally, and the CTLA4 composition can be or include CTLA4-Ig.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph that depicts the effects of CTLA4-Ig on IFN-γ production by ASD, DPI, and control PBMCs in response to β-lactoglobulin (10 μg/ml) for 4 days with or without CTLA4-Ig (10 μg/ml).

FIG. 2 is a graph that depicts the effect of CTLA4-Ig on TNF-α production by ASD, DPI, and control PBMCs in response to β-lactoglobulin. PBMCs were incubated with β-lactoglobulin (10 μg/ml) for 4 days with or without CTLA4-Ig (10 μg/ml).

### DETAILED DESCRIPTION

The invention is based, in part, on the discovery that CTLA4-Ig can be used to treat ASD, and particularly to treat ASD children with DPI. One study on which the invention is based (see Example 1) demonstrated the effects of CTLA4-Ig, a competitive inhibitor of CD28 signaling for T cell activation, on DP-triggered cytokine production by ASD, DPI, and control PBMCs. The results obtained indicate that blocking CD28 signaling partially suppresses IFN-γ production by PBMCs, but not production of TNF-α, IL-5, or IL-12p40 following stimulation with DPs.

A second study (Example 2) indicates that CTLA4-Ig is safe to administer to human patients. Example 3 describes a treatment regimen for a patient with ASD.

Autism, Dietary Protein Intolerance, and CTLA4-Ig

Delayed-type, non IgE-mediated immune responses against DPs have been implicated in the pathogenesis of GI symptoms observed in ASD children. IgG antibodies, cytokines produced by T cells and other lineage cells, and direct cytotoxic T cell actions may be involved in non-IgE mediated immune responses against DPs (Sampson and Anderson, J. Ped. Gastroenterol. Nutr., 30:S87-S94, 2000). In non IgE-mediated immune reactions to DPs, production of inflammatory cytokines, especially TNF-α in the gut mucosa, appears to play a role in the pathological effect of a reaction (Dupont and Heyman, J. Ped. Gastroenterol. Nutr., 30:S50-7, 2000; Heyman et al., Gastroenterology, 106:1514-23, 1994; Adams et al., J. Immunol., 150:2356-63, 1993; Terpend et al., Gut, 42:538-45, 1998). In DP-sensitive enteropathy, local immune reactions to DPs in the gut induce inflammatory cytokines, leading to chronic gut inflammation and an increase in gut permeability to macromolecules (Dupont and Heyman, 2000, supra; Benlounes et al., Clin. Exp. Allergy, 27:942-8, 1997). This may further lead to sensitization to other DPs, resulting in multiple DPI. In ASD patients, increased gut permeability (D'Eufemia et al., Acta Pediatr., 85:1076-9, 1996) and disaccharide malabsorption (Horvath et al., J. Pediatr., 135:559-63, 1999) have also been reported.

DPI patients have circulating PBMCs secreting TNF-α/IFN-γ against DPs (Benlounes et al., J. Allergy Clin. Immunol., 104:863-9, 1999; Dupont and Heyman, supra; Benlounes et al., 1997, supra; Heyman et al., 1994, supra). In these patients PBMCs produce elevated levels of TNF-α and IFN-γ in response to DPs (CMP, gliadin, and soy protein) at high frequency, as observed in patients with CMP intolerance (Benlounes et al., 1999, supra). Among CMPs, β-lactoglobulin was the most potent in inducing TNF-α and IFN-γ production by ASD PBMCs and those from patients of DPI (see Example 1, Tables 1 and 3). It is estimated that less than 1 out of 10⁴⁻⁵ naïve T cells will respond to protein antigen such as ovalbumin (Bevan and Goldrath et al., Curr. Biol., 10:R338-40, 2000). Thus, DP-specific effector and/or memory T cells may be more prevalent in ASD and DPI patients than in healthy individuals.

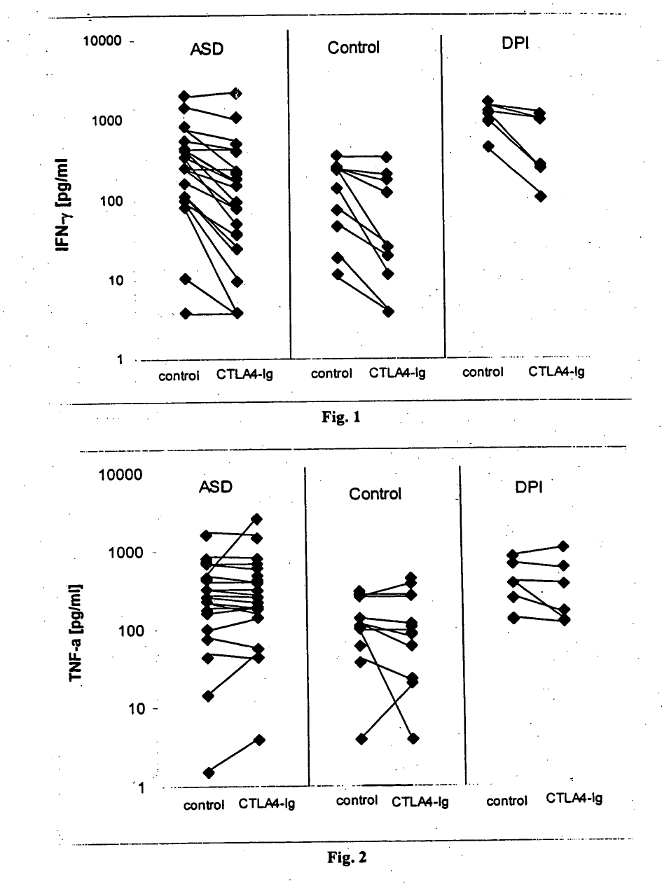

T cell activation by protein antigen is regulated by two signaling mechanisms: one through TCR-recognizing antigen presented by MHC molecules; and the other by co-stimulatory molecules. CD28 and its ligands are crucial co-stimulatory molecules for naïve T cells, but memory T cells may be less dependent on CD28-mediated signaling (Gross et al., J. Immunol., 149:380-8, 1992; Chambers, Trends Immunol., 22:217-23, 2001), and more dependent on signaling mediated by ICOS, a receptor believed to be involved in the stimulation of previously activated T cells (Sporici and Perrin, Clin. Immunol., 100:263-9, 2001). Example 1 (below) demonstrates that PBMCs produced less IFN-γ in response to β-lactoglobulin when CTLA4-Ig, a competitive inhibitor of CD28 signaling, was supplemented to the cultures (see FIG. 1). Therefore, cytokine production by PBMCs in response to DP stimulation is at least partially dependent on CD28-mediated T-cell activation. The effect of CTLA4-Ig was proportionally similar in control, ASD, and DPI PBMCs, although total IFN-γ production was greater in ASD and DPI PBMCs with β-lactoglobulin. Inhibitory action of CTLA4-Ig was less remarkable with other antigens (soy, casein, and MBP). This was mainly reflective of low baseline production of IFN-γ with these stimuli. DPI PBMCs produced high levels of IFN-γ and this was partially blocked by CTLA4-Ig (see Table 1). CTLA4-Ig did not suppress IL-5 production, a representative T2 cytokine. This was also likely due to low baseline IL-5 production in response to DPs.

Protein antigens, including DPs, are regarded as potent stimulants for T cells, but not for other lineage cells. Thus, the type 1 T (T1) cells are likely a major source of IFN-γ produced in response to DPs. The results thus indicated that DP specific T1 cells in both ASD and DPI PBMCs are partially dependent on CD28 signaling in a similar manner.

PBMCs produced significant amounts of IL-12p40 in response to DPs, especially β-lactoglobulin (see Example 1, Table 4). ASD and DPI PBMCs produced elevated amounts of TNF-α with β-lactoglobulin (Example 1, Table 3). There was little suppressive action of CTLA4-Ig on TNF-α and IL-12p40 production by PBMCs in all the study groups (Table 4). As opposed to IFN-γ, TNF-α production following stimulation with DPs was more likely to occur by various lineage cells including macrophage and monocyte lineage cells and T cells; the presence of TNF-α+ T cells was reported in patients with rheumatoid arthritis and inflammatory bowel disease (Mariani et al., Digest Dis. Sci., 45:2029-35, 2000; Steiner et al., Rheumatology, 38:202-13, 1999). Macrophage and monocyte lineage cells were also likely the major source of IL-12p40 with stimuli of DPs (Jankovic et al., TRENDS in Immunol., 22:450-7, 2001). Activated T cells produce various cytokines that in turn activate macrophage and monocytes to produce TNF-α and IL-12. DP-induced TNF-α and IL-12p40 production by PBMCs may be partly explained by this mechanism, in addition to TNF-α production by activated T cells. Alternatively, it may be possible that some protein component can stimulate other lineage cells in an antigen-independent manner inducing TNF-α and IL-12p40 production.

In summary, the invention is based, in part, on the discovery of partial inhibitory effects of CTLA4-Ig on DP-triggered IFN-γ production by ASD and DPI PBMCs. In addition to an elimination diet, partial inhibition of IFN-γ and TNF-α production against DP can be used to therapeutically treat symptoms of ASD, including DPI observed in ASD children.

Use of CTLA4-Ig in Treating Patients with ASD

The invention provides new methods of treating patients who have symptoms of ASD, for example, symptoms of DPI, by administering an effective amount of a CTLA4 composition. The methods are also useful to treat symptoms of DPI in patients not diagnosed with ASD. An “effective amount of a CTLA4 composition” is an amount of the composition that alleviates at least one symptom of ASD or DPI for at least an hour or more (e.g., 3, 6, 12, 18, or 24 hours or more), and to an extent that can be determined by using standard diagnostic methods.

Patients with ASD can present one or more of at least the following gastrointestinal symptoms: GI cramping, chronic diarrhea, chronic constipation, gastroesophageal reflux (GER), bloating, colic, vomiting, and related indications. These GI symptoms can result from DPI, which presents similar indications in response to particular dietary proteins. The most common dietary proteins implicated in DPI include the cow's milk proteins β-lactoglobulin (most common cause of DPI), alpha-lactoglobulin, casein, bovine serum albumin, and γ-lactoglobulin; soy protein; gliadin, a protein present in wheat flour; and beef and lamb-based formulas. A patient diagnosed with ASD, including one who also presents with DPI, can be treated with an amount of a CTLA4 composition effective to lessen one or more of the above GI symptoms.

Symptoms of ASD are described in the DSM-IV guidelines and include impairment in social interaction, manifested by marked impairment of non-verbal behaviors such as eye-to-eye gaze, facial expression, body postures and gestures to regulate social interaction. Symptoms can also include failure to develop peer relationships appropriate to developmental level, lack of spontaneous seeking to share enjoyment, interests, or achievements with other people (e.g., by a lack of showing, bringing, or pointing out objects of interest), and/or lack of social or emotional reciprocity. Other symptoms include impairment in communication marked by a delay in, or total lack of, the development of spoken language (not accompanied by an attempt to compensate through alternative modes of communication such as gesture or mime).

In individuals with adequate speech, an ASD child may display marked impairment in the ability to initiate or sustain a conversation with others, stereotyped and repetitive use of language or idiosyncratic language, and/or lack of varied, spontaneous make-believe play or social imitative play appropriate to his or her developmental level. Restricted, repetitive and stereotyped patterns of behavior, interests, and activities can also be symptoms of ASD. These symptoms may be marked with a preoccupation with one or more stereotyped and restricted patterns of interest that is abnormal either in intensity or focus; an apparently inflexible adherence to specific, nonfunctional routines or rituals; stereotyped and repetitive motor mannerisms (e.g., hand or finger flapping or twisting, or complex whole-body movements); and/or a persistent preoccupation with parts of objects.

ASD children may also experience delays or abnormal functioning in at least one of the following areas, with onset prior to age 3 years: (1) social interaction, (2) language as used in social communication, or (3) symbolic or imaginative play.

ASD children can also exhibit elevated levels of cytokines, such as in response to ingestion of particular dietary proteins. Elevated levels of the T cell cytokine IFN-γ following ingestion of DPs can be a symptom of ASD. The elevated cytokines can also include the inflammatory cytokines, TNF-α, IL-1β, IL-2, IL-5, IL-6, IL-8, IL-10, IL-12, IL-12p40, or MIP-1α, for example.

According to the methods of the invention, an effective amount of a CTLA4 composition will improve (lessen the severity of) at least one of the ASD symptoms described above. One useful form of CTLA4 is CTLA4-Ig. The CTLA4 can be isolated from natural sources, can be generated using standard techniques, such as by engineered yeast, bacteria, or mammalian cells, or can be synthesized.

CTLA4-Ig is a CTLA4-immunoglobulin fusion protein having a modified immunoglobulin constant region which may be modified to reduce at least one constant region-mediated biological effector function, thereby having improved immunoinhibitory properties. The CTLA4-Ig fusion protein can inhibit an interaction between a CTLA4 ligand (CD80 and/or CD86, for example) on an antigen presenting cell and a receptor for the CTLA4 ligand (CD28 and/or CTLA4, for example) on the surface of T cells to thereby suppress an immune response in the subject. The CTLA4 component of the CTLA4-Ig fusion protein can have at least one CTLA4 activity, e.g., the ability to bind to the natural ligand(s) of the CTLA4 antigen on immune cells, and to inhibit or interfere with immune cell mediated responses. The CTLA4 component can be an extracellular domain of the CTLA4 protein. Any CTLA4 for use in the CTLA4 compositions described herein must have at least one activity of wildtype CTLA4.

The immunoglobulin constant region can be modified to reduce at least one Ig constant region-mediated biological effector function. The Ig constant region can consist of a hinge region, a CH2 domain and a CH3 domain derived from Cγ1, Cγ2, Cγ3, or Cγ4. The constant region can be altered (e.g., mutated at specific amino acid residues by substitution, deletion or addition of amino acid residues) to reduce at least one IgC region-mediated effector function. The CH2 domain of the Ig region can be modified to reduce a biological effector function, such as complement activation and/or Fc receptor interaction.

CTLA4 compositions can be safely administered to subjects, such as a human subject (a child with ASD, for example) at a range of doses. For example, CTLA4-Ig can be administered at 0.5, 1.5, 5.0 or 10.0 mg/kg. Doses of CTLA4-Ig ranging from about 0.1 to 10.0 mg/kg can be effective in lowering the concentration of IFN-γ in patients with ASD, including those who also have DPI, which, in turn, can reduce symptoms of these disorders. For example, an effective dose of CTLA4-Ig for a 50 kg patient can be between about 5 and 500 mg administered once a day. By comparison, the corresponding effective doses of CTLA4-Ig for 30, 40, and 60 kg patients can be between about 3 and 300 mg per day, 4 and 400 mg per day, and 6 and 600 mg per day, respectively. Generally, the dosage of CTLA4 in a CTLA4 composition will not exceed 10.0 mg/kg. However, doses either higher or lower than these general ranges can be appropriate depending on the particular physiological characteristics or severity of symptoms of the patient.

Although the magnitude and frequency of doses of CTLA4 can vary, it is important that whatever regimen is employed, the amount of CTLA4 in the blood must be maintained at a level effective to lower the amount of IFN-γ.

Administration of CTLA4 compositions can be carried out over a range of time periods. It can be administered daily, once every few days, weekly, or monthly. The timing of administration varies from patient to patient, depending on such factors as the severity of a patient's ASD or DPI symptoms, the level of IFN-γ in a patient, and the duration of an effective concentration of CTLA4 in a patient. Often, it will be unnecessary to administer CTLA4 to a patient more frequently than once a week. For example, an effective dose of CTLA4 can be administered to a patient once a month for an indefinite period of time, or until the patient no longer requires therapy. In addition, sustained release compositions containing CTLA4 be used to maintain a relatively constant dosage in the patient's blood.

Pharmaceutical compositions containing CTLA4 for use in accordance with the present invention may be formulated in a conventional manner using one or more physiologically acceptable carriers or excipients known in the art. Acceptable excipients are those capable of preserving the activity of CTLA4 protein.

Compositions and their physiologically acceptable salts and solvates may be formulated for parenteral administration. For example, CTLA4 can be administered subcutaneously in the following formulation: 10 mg/mL CTLA4, e.g., CTLA4-Ig, 150 mM NaCl, 20 mM potassium phosphate, 0.01% polysorbate 20.

CTLA4 can be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

Routes of administration can include, but are not restricted to, intravenous, intramuscular, subcutaneous, intradermal, or transdermal administration, inhalation, or insufflation (either through the mouth or the nose). Other routes of administration can include intracranial, intraorbital, opthalmic, intraventricular, intracapsular, intraspinal, intracisternal, intraperitoneal, or transmucosal.

In addition to the formulations described previously, the compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compositions for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, for example, gelatin, for use in an inhaler or insufflator may be formulated containing a powder mix of the compound and a suitable powder base such as lactose or starch.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The compositions can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

The compounds may also be formulated in rectal compositions such as suppositories or retention enemas, for example, containing conventional suppository bases such as cocoa butter or other glycerides.

The invention is further illustrated by the following examples, which should not be construed as further limiting.

### EXAMPLES

### Example 1

### Effect of CTL4-Ig on Cytokine Production by PBMCs of ASD Children

The invention is based, in part, on studies involving ASD children suffering developmental regression [N=20, Age: median 5.4 years, (2-15 years) Sex; 4 females and 16 males]. Six adult patients with DPI and eleven healthy adults were included in the study. Diagnosis was ascertained according to the guidelines set forth in the Diagnostic and Statistical Manual of Mental Disorders, Fourth Edition (DSM-IV), and/or criteria established by the International Classification of Diseases (ICD-10; World Health Organization), Autistic Diagnostic Interview-Revised (ADI-R) and/or Autistic Diagnostic Observational Schedules (ADOS). Professionals specialized in behavioral/developmental pediatrics performed the studies described herein.

Most ASD children (18/20) recruited to the study were reported by parents, teachers, and/or therapists to have clinical improvements with a gluten-free/casein-free (gf/cf) diet. DPI was diagnosed using the following criteria: i) onset of various GI symptoms (colic, diarrhea, GI cramping, vomiting, etc.) several hours after ingestion of proteins, wherein the immediate symptoms were not associated with an IgE-mediated immune response; ii) absence of symptoms when causative DPs were avoided; and iii) recurrence of symptoms when causative DPs were reintroduced into the diet.

Elevated IgE levels (>100 IU/ml), presence of allergen-specific IgE antibody (Ab), and/or positive reactivity to prick skin test (PST) was reported in 5/20 (25%) ASD patients; the frequency of atopy is equivalent to that observed in healthy siblings (5/26, 23%) as reported in previous studies and in a general population (Ono, Annu. Rev. Immunol., 8:347-66, 2000). Anti-MBP (myelin basic protein) antibodies were detected in 10/20 ASD patients. Peripheral blood (PB) samples were obtained when the study subjects were afebrile, free of antibiotics or steroids, and free from symptoms indicating viral syndrome or other common childhood infections. All the ASD patients had either never been on steroid or were steroid-free for at least four weeks prior to PB collection. In addition, ASD patients had never been treated with secretin, or had not been treated with secretin for at lease six weeks prior to PB collection. All the ASD children in the study followed a gf/cf diet for at least three months prior to the start of the study.

For cell preparations and cultures, peripheral blood was obtained by venipuncture of cubic veins, and coincided with routine blood work in most ASD patients. Peripheral blood mononuclear cells (PBMCs) were obtained by Ficoll-Hypaque density gradient and cultured as described previously (Jyonouchi et al., Arch. Otolaryngol. Head Neck Surg., 126:522-8, 2000).

Assessment of Immune Responses

Immune responses against dietary proteins were assessed by measuring cytokine production by PBMCs following exposure to dietary protein stimulants (also called antigens below). Stimulants included soy protein extracts (provided by Ross Products Division/Abbott Laboratories, Columbus, Ohio), bovine casein (Sigma-Aldrich Corp., St. Louis, Mo.), and β-lactoglobulin (Sigma-Aldrich Corp., St. Louis, Mo.). Less than 1 ng/ml endotoxin was detected in these dietary proteins (1 mg/ml solution; Endotoxin kit, Sigma-Aldrich Corp., St. Louis, Mo.).

PBMCs from ASD children were found to react to β-lactoglobulin with the highest frequency and to casein with a lower frequency. Reactivity to gliadin was observed fairly frequently in ASD PBMCs (in samples collected from about 50% of ASD subjects), but this reactivity was rare in DPI patients. Therefore, the suppressive effects of CTLA4-Ig on gliadin-induced T cell cytokine production was not tested in the current study. Most ASD children in the study were reported to be positive for anti-MBP IgG antibodies, and, therefore, MBP (Sigma-Aldrich Corp., St. Louis, Mo.) was used as an antigen and also as a recall antigen. Stimulants were used in concentrations according to previous studies (Jyonouchi et al., J. Neuroimmunol., 120:170-9, 2001; Jyonouchi et al., Arch. Otolaryngol. Head Neck Surg., 126:522-8, 2000). CTLA4-Ig (provided by RepliGen Corp., Needham, Mass.) was dissolved into PBS and aliquoted (11 mg/ml). The final concentration of CTLA4-Ig used was 10 μg/ml.

IFN-γ and IL-5 were selected as representative T1 and T2-cytokines, respectively. IL-4, and IL-2 were also measured in a few study subjects and control adults. However, no significant production of IL-2 or IL-4 with DPs was observed, and therefore, this line of investigation was not followed further. Macrophage and monocyte lineage cells are generally regarded as a major source of TNF-α, and they do not respond to soluble protein antigens such as DPs. However, other lineage cells, including T cells, can produce TNF-α (Bregenholt and Claesson, Eur. J. Immunol., 28:379-89, 1998; Trobonjaca et al., J. Immunol., 166:3804-12, 2001), and activated T cells can in turn induce TNF-α production by macrophages/monocytes through IFN-γ production. This study measured TNF-α production, as well as IL-12p40 as a marker for monocyte/macrophage activation by activated T cells.

PBMCs were cultured with stimulants for four days, and then PBMC supernatants were collected. Cytokine levels in the culture supernatants were measured by enzyme-linked immunosorbent assay (ELISA). OptEIA™ Reagent Sets (BD Pharmingen, San Diego, Calif.) were used to measure levels of IFN-γ, TNF-α, IL-12p40, and IL-5. Appropriately diluted samples were used for the ELISA assays according to Jyonouchi et al. (J. Neuroimmunol., 120:170-9, 2001). Samples were assayed at an optical density of 450 nm to determine cytokine concentrations. Intra- and inter-variations of cytokine levels were less than 5%.

The Mann-Whitney test or the Wilcoxon weighed ranks test was used to evaluate the equality of two sets of independent samples, or two sets of related samples, respectively. The Kruskal-Wallis test was used to compare multiple values. The two-tailed Kendall τ-b test was used to correlate two different parameters. Differences with p<0.05 were considered to be significant.

As shown in Table 1, below, both ASD and DPI PBMCs produced higher amounts of IFN-γ than did control PBMCs in response to β-lactoglobulin; 45% (9/20) ASD PBMC samples and 100% (6/6) DPI PBMC samples produced >2 standard deviation (SD) above the control mean (CM) (>2 SD+CM) values of IFN-γ.

In response to soy protein, 66.7% (4/6) DPI PBMC samples produced >2 SD+CM values of IFN-γ. CTLA4-Ig suppressed IFN-γ production by PBMCs significantly in all groups when β-lactoglobulin was used as a stimulant. FIG. 1 illustrates the effects of CTLA4-Ig on IFN-γ production by ASD, DPI and control PBMCs in response to β-lactoglobulin. Data points illustrate an overall decrease in IFN-γ levels in the presence of CTLA4-Ig. In particular, in 60% (12/20) ASD children, 50% (3/6) DPI subjects, and 82% (9/11) control subjects, the level of IFN-γ produced by PBMCs was decreased by <40% in the presence of CTLA4-Ig.

TABLE 1
IFN-γ levels (pg/mL)¹ in PBMCs following stimulation with dietary protein (DP) antigens

| | ASD children | DPI patients | Control subjects |
|---|---|---|---|
| DP Antigen | (N = 20) | (N = 6) | (N = 11) |
| Soy | 135.4 (<3.9-1496) | 632.4 (289.9-1562)³ | 113.9 (<3.9-350.9) |
| Soy + CTLA4-Ig | 18.6 (<3.9-2903) | 421.2 (104.5-803.3)^{2,3} | 18.9 (<3.9-207.7) |
| Casein | 17.9 (<3.9-2512) | 171.5 (20.5-427.4)4 | 25.6 (<3.9-357.7) |
| Casein + CTLA4-Ig | <3.9 (<3.9-2251)² | 11.0 (<3.9-216.5) | <3.9 (<3.9-151.9) |
| β-lactoglobulin | 303.3 (<3.9-2057) | 1081 (449.1-1652.4)³ | 143.7 (11.6-370.2) |
| β-lactoglobulin + CTLA4-Ig | 88.4 (0.8-2207)² | 642.1 (106.1-1189)^{2,3} | 20.3 (<3.9-346.6)² |
| MBP | <3.9 (<3.9-786.1) | 79.5 (7.7-1155) | <3.9 (<3.9-33.9) |
| MBP + CTLA4-Ig | <3.9 (<3.9-2529) | <3.9 (<3.9-599.4) | <3.9 (<3.9-8.9) |

¹The results were expressed as median (range indicated in parenthesis).
²Values are significantly lower in the presence of CTLA4-Ig (10 μg/ml) than in the absence of CTLA4-Ig (p < 0.05).
^{3,4}Values are significantly higher than cytokine levels produced by control PBMCs; (p < 0.005)³ and (p < 0.05)⁴.

In contrast, when soy protein was used as the stimulant, PBMCs from 100% (6/6) DPI subjects, demonstrated a decrease in IFN-γ by >40% in the presence of CTLA4-Ig (see Table 1). When either casein or MBP were used to stimulate the PBMCs, the suppressive action of CTLA4-Ig appeared on IFN-γ production was not evident partly due to production of low levels of IFN-γ by PBMCs with these stimuli in all the groups.

TABLE 2
IL-5 levels (pg/mL)¹ in PBMCs following stimulation
with dietary protein (DP) antigens

| | ASD children | DPI patients | Control subjects |
|---|---|---|---|
| DP Antigen | (N = 20) | (N = 6) | (N = 11) |
| Soy | 68.8 (<3.9-377.3) | 59.2 (<3.9-85.3) | 12.2 (<3.9-207) |
| Soy + CTLA4-Ig | 18.3 (<3.9-136.6) | 31.1 (<3.9-87) | 5.6 (<3.9-128.3) |
| Casein | <3.9 (<3.9-92.0) | 12.4 (2.7-93.6) | <3.9 (<3.9-92.0) |
| Casein + CTLA4-Ig | <3.9 (<3.9-116.8) | <3.9 (<3.9-90.3) | <3.9 (<3.9-116.8) |
| β-lactoglobulin | <3.9 (<3.9-113.5) | <3.9 (<3.9-82) | <3.9 (<3.9-128.3) |
| β-lactoglobulin + CTLA4-Ig | <3.9 (<3.9-182.9) | <3.9 (<3.9-82) | <3.9 (<3.9-136.6) |
| MBP | 9.5 (<3.9-108.5) | <3.9 (<3.9-130.0) | <3.9 (<3.9-103.5) |
| MBP + CTLA4-Ig | 12.5 (<3.9-219.3) | <3.9 (<3.9-83.7) | <3.9 (<3.9-128.3) |

¹The results were expressed as median (range indicated in parenthesis).
²Values are significantly lower in the presence of CTLA4-Ig (10 μg/ml) than in the absence of CTLA4-Ig. P values are shown in parenthesis below the two concentration values in each box (p < 0.05)
^{3,4}Values are significantly higher than cytokine levels produced by control PBMCs; (p < 0.005)³ and (p ≦ 0.05)⁴.

As observed in Table 2, PBMCs from all the study subjects produced little IL-5 in response to these recall antigens, and CTLA4-Ig did not alter IL-5 production except for its suppressive effects in ASD PBMCs stimulated with soy protein; 11/20 ASD PBMCs produced >40% less IFN-γ with a stimulus of soy protein.

PBMCs from ASD and DPI subjects produced higher amounts of TNF-α in response to β-lactoglobulin than did control PBMCs (see Table 3); 50% (10/20) ASD PBMC samples and 100% (3/3) DPI PBMC samples produced >2 SD+CM values of TNF-α.

TABLE 3
TNF-α levels (pg/mL)¹ in PBMCs following stimulation with DP antigens

| | ASD children | DPI patients | Controls |
|---|---|---|---|
| DP Antigen | (N = 20) | (N = 6) | (N = 11) |
| Soy | 110.3 (3.6-312.1) | 67.7 (26.5-187.6) | 30.7 (<3.9-291.9) |
| Soy + CTLA4-Ig³ | 75.0 (2.1-5665) | 59.2 (13.3-221.3) | 21.6 (<3.9-243.1) |
| Casein | 31.4 (<3.9-669.1) | 40.4 (<3.9-344.1) | 18.2 (<3.9-268.4) |
| Casein + CTLA4-Ig | 24.8 (<3.9-5229) | 22.5 (<3.9-154.0) | 12.3 (<3.9-349.2) |
| β-lactoglobulin | 321.0 (1.5-1598)² | 304.0 (126-825.6)² | 101.4 (<3.9-288.7) |
| β-lactoglobulin + CTLA4-Ig | 281.6 (<3.9-2562)² | 263.2 (117.6-1063)² | 87.2 (<3.9-430.9) |
| MBP | 25.4 (<3.9-329.1) | 17.6 (14-132.1) | <3.9 (<3.9-266.7) |
| MBP + CTLA4-Ig | 27.0 (<3.9-5248) | 43.2 (8.5-152.3) | <3.9 (<3.9-185.9) |

¹The results are expressed as median (range indicated in parenthesis).
²Values are significantly higher than values produced by control PBMCs (p < 0.05).
³Concentration of CTLA4-Ig was 10 μg/ml.

CTLA4-Ig did not suppress TNF-α production by PBMCs in any of the three test groups, regardless of which stimulant was used (see Table 3 and FIG. 2). FIG. 2 illustrates the general lack of any effect of CTLA4-Ig on TNF-α production by ASD, DPI and control PBMCs in response to β-lactoglobulin. IL-12p40 production by DPI and ASD PBMCs was also relatively similar to control PBMCs when each of the stimulants was tested. Generally, higher IL-12p40 production was observed in response to treatment with β-lactoglobulin, than with soy, casein, or MBP (see Table 4). Of the β-lactoglobulin treated subjects, the DPI PBMCs produced the highest amount of IL-12p40 (see Table 4). However, only one out of six DPI PBMC samples produced >2 SD+CM values of this cytokine. CTLA4-Ig did not significantly alter IL-12p40 production by PBMCs in any of the groups.

TABLE 4
IL-12p40 levels (pg/mL)¹ in PBMCs following stimulation with DP antigens

| | ASD children | DPI patients | Controls |
|---|---|---|---|
| DP Antigen | (N = 20) | (N = 6) | (N = 11) |
| Soy | 63.7 (<7.8-1027) | 354.5 (<7.8-890.9) | 16.1 (<7.8-755.4) |
| Soy + CTLA4-Ig³ | <7.8 (<7.8-2903) | 357.3 (<7.8-528.3) | <7.8 (<7.8-977.6) |
| Casein | 258.6 (<7.8-2512) | 198.8 (<7.8-1759) | 312.8 (<7.8-1052) |
| Casein + CTLA4-Ig | 262.0 (<7.8-2251) | 216.5 (<7.8-1360) | 338.6 (<7.8-1003) |
| β-lactoglobulin | 983.6 (120.1-2730) | 1634 (1122-2612)² | 1070 (171.3-2738) |
| β-lactoglobulin + CTLA4-Ig | 1128 (147.3-2607) | 1185 (484.0-2145) | 754.8 (57.2-2779) |
| MBP | 10.0 (<7.8-1916) | 11.4 (<7.8-309.2) | <7.8 (<7.8-591.0) |
| MBP + CTLA4-Ig | 11.4 (<7.8-574.4) | 9.5 (<7.8-1131) | <7.8 (<7.8-574.4) |

¹The results are expressed as median (range indicated in parenthesis).
²Values are significantly higher than values produced by control PBMCs (p < 0.05).
³Concentration of CTLA4-Ig was 10 μg/ml.

In summary, PBMCs produced less IFN-γ in response to β-lactoglobulin when CTLA4-Ig, a competitive inhibitor of CD28 signaling, was supplemented to the cultures. Thus, cytokine production by PBMCs in response to DP stimulation is at least partially dependent on CD28-mediated T-cell activation, and as a result, CTLA4 compositions can be used to inhibit DPI symptoms. In addition, PBMCs produced significant amounts of IL-12p40 in response to DPs, especially β-lactoglobulin, and ASD and DPI PBMCs produced elevated amounts of TNF-α with β-lactoglobulin. While there was relatively little suppressive action by CTLA4-Ig on TNF-α and IL-12p40 production by PBMCs in these study groups, this was expected, because a number of different cells produce these cytokines, whereas CTLA4 preferentially inhibits cytokine production in T cells.

More specifically, unlike IFN-γ production, which occurs primarily in T cells, TNF-α production following stimulation with DPs is more likely to occur in a variety of cells including macrophages, monocytes, and T cells. For example, the presence of TNF-α+ T cells was reported in patients with rheumatoid arthritis and inflammatory bowel disease (Mariani et al., Digest Dis. Sci., 45:2029-35, 2000; Steiner et al., Rheumatology, 38:202-13, 1999). Macrophage and monocyte lineage cells are also likely the major source of IL-12p40 after stimulation by DPs (Jankovic et al., TRENDS in Immunol., 22:450-7, 2001). Activated T cells produce various cytokines that in turn activate macrophage and monocytes to produce TNF-α and IL-12. DP-induced TNF-α and IL-12p40 production by PBMCs may be partly explained by this mechanism, in addition to TNF-α production by activated T cells.

It is also known that protein antigens, including DPs, are regarded as potent stimulants for T cells, but not for other cells. Thus, the type 1 T (T1) cells are likely the major source of IFN-γ produced in response to DPs. Taken together, all of these results indicate that CTLA4 compositions can be administered to ASD and/or DPI subjects to partially inhibit IFN-γ and TNF-α production induced by DPs, and will thus treat or ameliorate symptoms of ASD and/or DPI.

### Example 2

### Phase I Study to Assess the Safety of Multiple Doses of CTLA4-Ig in Healthy Normal Volunteers

A phase I study was conducted at the PPD Development Clinic (“Clinic”), 72 Hospital Close, Evington, Leicester, LE5 4WW, UK. The primary objection was to evaluate the safety of CTLA4-Ig, in particular, CTLA4-IgG4m, which has the product code RG2077. RG2077 was administered via intravenous (i.v.) infusion on three consecutive days to healthy male volunteers. A secondary objective was to observe the effect of RG2077 (provided by RepliGen Corp., Needham, Mass.), administered via i.v. infusion on three consecutive days, on the immune function of healthy male volunteers. RG2077 contains a CTLA4-Ig fusion protein consisting of the extracellular domain of human CTLA4 (amino acid residues 1-125), and has reduced Fc receptor interaction due to two amino acid substitutions in the CH2 domain of the immunoglobulin peptide (substitution of leucine at position 235 with glutamate and substitution of glycine at position 237 with alanine.

The study was a single-center, single-blind, placebo-controlled, dose escalation safety study. Sixteen healthy male volunteers were enrolled in the study. They ranged in age from 18 to 55 years and had body mass indices between 18 and 28 kg/m². Bodyweight was <80 kg. Volunteers had not received treatment with any immunosuppressant agent within 1 year prior to the study, nor with any antibody treatment within 6 months prior to the start of the study.

The volunteers were divided into four groups. Subjects were admitted to the Clinic on the evening before dosing (Study Day −1). Subjects in Groups 1 to 4 received RG2077 at 0.5, 1.5, 5.0 and 10.0 mg/kg/day, respectively, on Study Days 1, 2 and 3. Subsequent groups were dosed after all four subjects in the previous group had undergone assessment on Day 7. Dose administration to the four individuals of each group was randomized so that three subjects received RG2077, and one received placebo. RG2077 and placebo were administered by i.v. infusion over 1 hour. Each subject also received an injection of keyhole limpet hemocyanin (KLH; 1.0 mg) 75 minutes after the start of the infusion on Day 1. A blood sample was collected at 33 hours post-third infusion on Day 4, and then the subject was discharged from the Clinic. Subjects attended out-patient visits at the Clinic on Days 5 to 10 (attendance varied depending on dose) and on Day 17, for assessments and/or to provide blood samples for pharmacokinetic (PK) analysis. All four subjects in a group underwent a safety assessment on Day 7 and then enrollment began for the next dose level. On Day 28 (±2 days), subjects attended the Clinic for post-study assessments and a second injection of KLH (0.1 mg). Subjects returned for a final visit three days after Day 28 (Day 31 [±2 days]).

Administration of the test drug, RG2077 (CTLA4-IgG4m; batch number RN000704) occurred as follows: 0.5, 1.5, 5.0, and 10.0 mg/kg, administered as a 70 mL (±20 mL) i.v. infusion over 1 hour on 3 consecutive days (Days 1, 2, and 3). Keyhole limpet hemocyanin (Immucothel®, biosyn Arzneimittel GmbH; Lot: 023443 036569) was administered in a 1.0 mg dose administered intradermally over the deltoid on Day 1, and in a 0.1 mg dose administered to the forearm on Day 28 (±2 days). The reference product, saline (batch number 01C05BK), was administered as a 70 mL (±20 mL) i.v. infusion over 1 hour. Like RG2077, saline was administered on 3 consecutive days.

The duration of the study was approximately 2 months, including the 30 day pre-study screening interval.

To evaluate efficacy of treatment, blood samples were collected before infusion on Days 1 to 3 and the minimum plasma concentration (Cmin) of RG2077 was determined. Blood samples were collected 65 minutes after the start of infusion on Days 1 and 2 to determine the maximum plasma concentration (Cmax) of RG2077. Blood samples were collected at time intervals relative to the third infusion (Day 3) for pharmacokinetic analysis of RG2077. Following administration of a 0.5 mg/kg pre-dose, blood was collected at 65, 80, 120, 180, 300, and 480 minutes, 25, 33, 49, 57, and 73 hours, and 7 and 14 days. Following administration of a 1.5 mg/kg pre-dose, blood was collected at 65, 80 120, 180, 300, and 480 minutes, 25, 33, 49, 57, 73, 85, and 97 hours, and 7 and 14 days. Following administration of a 5.0 mg/k pre-dose, blood was collected at 65, 80 120, 180, 300, and 480 minutes, 25, 33, 49, 57, and 73 hours, and 4, 5, 6, 7, and 14 days. Following administration of a 10.0 mg/kg pre-dose, blood was collected at 65, 80 120, 180, 300, and 480 minutes, 25, 33, 49, 57, and 73 hours, and 4, 5, 6, 7, and 14 days.

The following pharmacokinetic parameters were evaluated for each dose: Cmin; Cmax; area under the serum concentration versus time curve (AUC); and half-life (t^{1/2}). A variety of pharmacodynamic parameters were evaluated: immunogenicity (anti-RG2077) was measured on Days 1, 17 and 28; immune response to KLH was measured on Day −1 (baseline), Day 17, and Day 28; delayed-type hypersensitivity (DTH) skin reaction to KLH was measured on Day 31; and receptor saturation and distribution of cell types was measured on Day 1 (before infusion), Day 4 (25 hours after the start of the third infusion on Day 3), Day 7, and Day 28.

Safety was evaluated by measuring the following factors: baseline signs and symptoms; antigenicity, serum virology, and serum immunology at screening; electrocardiogram (ECG) at screening; haematology and clinical chemistry at screening and on Days −1, 7, 17, and 28; coagulation monitoring at screening and on Days 7 and 28; urinalysis at screening and on Day 28; physical examination at screening and on Days −1, 7, 17 and 28; vital signs (blood pressure, pulse and oral temperature) at intervals throughout the study; and adverse events (AEs) throughout the study.

An AE was defined in the protocol as any sign (including the clinical manifestation of abnormal laboratory results) or medical diagnosis noted by medical personnel, or any symptom reported by the subject, regardless of relationship to study drug, that: 1) had onset anytime after the start of study drug treatment, or 2) had worsened since the event was previously reported (this included worsening of signs, symptoms, or diagnoses that were ongoing at the start of study drug treatment). An AE was also defined in the protocol as any untoward medical occurrence in a subject administered a pharmaceutical product, which did not necessarily have a causal relationship with the treatment. An AE could be any unfavorable and unintended sign (e.g. including an abnormal laboratory finding), symptom, or disease temporally associated with the use of the study drug, whether or not it was considered to be drug related. This included any newly occurring event or previous condition that had increased in severity or frequency since the administration of the study drug. Subjects were monitored for AEs from the start of study drug treatment until Day 28 (±3 days), when all the required evaluations were complete. Adverse events were reviewed at all study visits. If the subject showed signs of adverse effects from an infusion, the observation period was extended until the event resolved. Subjects were interviewed in a non-direct manner to identify potential AEs. The occurrence of an AE was based on changes in the subject's physical examination, laboratory results and/or sign and symptoms. Adverse events were monitored until they had resolved or were clearly determined to be due to a subject's stable or chronic condition or intercurrent illness(es).

Statistical methods were used in the analysis of the clinical data. Adverse events were analyzed by treatment by examination of the number of events, number of subjects, and percentage of subjects, in terms of type of event/body system, severity and relationship. Shift tables were used to examine shifts in clinical laboratory values (haematology and blood chemistry) from baseline to each post-treatment time point, by treatment. The Stuart-Maxwell test or McNemar's test, as appropriate, was used to test the significance of shifts. In addition, changes in value from baseline within each treatment group were examined, and the Wilcoxon test was used to determine the significance of these changes.

Pharmacodynamic data was also gathered and evaluated. Before and 65 minutes after the start of dosing on Days 1, 2, and 3, the RG2077 blood levels were measured to determine Cmin and Cmax. Peak and trough drug levels for the four dose groups were determined. RG2077 blood levels were assessed 1, 2, 3, 4, 5, 6, 7, and 14 days post-third infusion. Non-parametric estimation of each individual's AUC and half-life decay parameters was performed, and the four study groups contrasted.

None of the subjects who received treatment with RG2077 experienced a delayed-type hypersensitivity (DTH) reaction when administered KLH. There were no serious adverse events (SAEs) reported during the study, and no withdrawals due to AEs. Twenty-nine treatment-emergent AEs were reported by 11 subjects. Four AEs were reported by subjects who received placebo, six AEs were reported by subjects who received 0.5 mg/kg/day RG2077, three were reported by subjects who received 1.5 mg/kg/day RG2077, eight were reported by subjects who received 5.0 mg/kg/day RG2077, and eight were reported by subjects who received 10 mg/kg/day RG2077. All reported AEs were considered to be mild in severity. A total of eight treatment-emergent AEs were considered to be unrelated to study drug, 10 AEs were considered unlikely to be related, and 11 were considered possibly related. Five of the 11 AEs considered to be possibly related to study drug were reported by subjects who received the 10 mg/kg/day dose. The most commonly reported AE was headache (six AEs). Coryza and nausea were each reported on three occasions, and lethargy was reported twice. All other AEs were reported only once. There were no individual clinically significant laboratory results during the study. There were also no notable changes in vital signs, ECG, laboratory results, or physical findings from pre- to post-dose or between dose groups. Over the studied period, RG2077 was considered to be safe and well tolerated in healthy male subjects, although a greater number of AEs considered possibly related to treatment were reported at the highest dose (10 mg/kg/day). The results indicate that CTLA4 compositions are safe for use in human ASD and DPI patients.

Primary Immune Response to KLH

CTLA4 plays a role in the physiological termination of antigen-specific immune responses. To investigate the effect of RG2077 on the immune response, study volunteers received an injection of keyhole lympet hemocyanin (KLH). Intradermal injection of 1 mg of KLH was performed one hour after the first dose administration of RG2077. The two major arms of the immune system, cellular and humoral, were assessed. Cellular immune responses were measured using a delayed-type hypersensitivity skin test, while humoral immunity to KLH was quantitated using an ELISA-based system to measure circulating levels of KLH-specific antibodies.

As part of the study, seroconversion of volunteers to KLH was assessed. Humoral immune responses to KLH were measured on study days 17 and 28. Antibody was detected using a sandwich ELISA where serial dilutions of serum samples were incubated on KLH-coated plates. Bound anti-KLH antibodies were detected with a goat anti human IgG, A, M-horseradish peroxidase (GαHIgGAM-HRP). The titer for each sample was determined to be the most dilute sample where the absorbance exceeded a value of 0.2. Seroconversion was defined as at least a four fold increase in titer at either day 17 or 28 as compared to the volunteers baseline titer, determined using the day 1 sample taken prior to KLH immunization.

The frequency of seroconversion was higher in the placebo group (2 of 4) than the frequency in volunteers receiving drug product (3 of 12) (see Table 5). An analysis of frequency by dose group is consistent with the notion that the two lowest dose groups did not blunt the humoral response to KLH (3 of 6) whereas the higher two dose groups were immunosuppressive (0 of 6) (see Table 5).

TABLE 5
Immune response against injected KLH or RG2077

| | # of KLH | # of DTH Skin | # of RG2077 |
|---|---|---|---|
| Group Number (dose) | seroconverted | Test Positives | seroconverted |
| Placebo | 2 of 4 | 2 of 4 | 0 of 0 |
| Drug Product (All) | 3 of 12 | 0 of 12 | 0 of 12 |
| 1 (0.5 mg/kg/day) | 1 of 3 | 0 of 3 | 0 of 3 |
| 2 (1.5 mg/kg/day) | 2 of 3 | 0 of 3 | 0 of 3 |
| 3 (5.0 mg/kg/day) | 0 of 3 | 0 of 3 | 0 of 3 |
| 4 (10.0 mg/kg/day) | 0 of 3 | 0 of 3 | 0 of 3 |

Delayed-type hypersensitivity (DTH) responses to KLH were measured by injecting 0.1 mg KLH intradermally on study day 28. The size of the subsequent tissue induration was measured 72 hours later.

The frequency of volunteers exhibiting a DTH skin test positive response to KLH was higher in the placebo group (2 of 4, 50%) (see Table 5). Volunteers 002 and 010 had tissue indurations that measured 13 and 8 mm, respectively. The sizes of these indurations were comparable to those observed in similar studies. In comparison, none of the volunteers receiving drug product had a measurable induration. In addition, volunteers 002 and 007 of the placebo group (2 of 4, 50%) seroconverted to KLH, while only 3/12 (25%) of the KLH recipients seroconverted.

These results demonstrate that the overall frequency of volunteers exhibiting an immune response directed against KLH was higher in the placebo group (3 of 4, 75%) as compared to the volunteers receiving drug product (3 of 12, 25%). The three KLH recipients who demonstrated an anti-KLH immune response received doses of the two lowest groups (0.5 and 1.5 mg/kg/day). These results indicate that CTLA4 compositions administered at a dose of 5 mg/kg can block primary immunization. The results are consistent with the role of CTLA4 as a terminator of antigen-specific immune responses.

CTLA4 Immunogenicity

Immunogenicity of CTLA4-Ig (RG2077) was determined using an ELISA-based assay system. Serum samples were collected from volunteers on study day −1 (baseline) and study days 17 and 28. An RG2077-coated plate was used to capture anti-RG2077 antibodies from the test samples. HRP-conjugated antibodies directed at human kappa and lambda chains were used to detect the captured anti-RG2077 antibodies. This approach allowed for the differentiation of anti-RG2077 antibodies in the serum sample and the Fc portion of the RG2077 on the coated plate. Volunteers administered placebo provided negative control data. A volunteer was determined to have seroconverted if either the day 17 or day 28 sample exhibited a four-fold increase in titer as compared to the baseline (day −1) sample. The lowest detectable titer in this system was 1:8.

None of the volunteers seroconverted to RG2077 at either time point post-infusion of drug product.

CTLA4 Half-Life In Vivo

Blood samples from test volunteers were analyzed for the presence of RG2077 at various time points during the course of the study. RG2077 concentrations were determined using a sandwich ELISA that employed a monoclonal capture antibody that recognizes CTLA4 and a detection antibody that binds human IgG4. Samples were quantitated using a standard curve generated with a reference standard RG2077.

A four week time point sample was obtained by using the samples collected for measuring the anti-KLH response. From these results, the half-life of RG2077 was calculated for each group. These values are shown in Tables 6 and 7. Table 6 shows the range of half-life values determined using a start window of 65 minutes to 24 hours whereas Table 7 shows the range of half-life values using a start window of 24 hours to the number given in the table. The corresponding correlation coefficient range (R²) is also provided.

TABLE 6
Half-life of RG2077 (65 minute Start time)

| Group (Treatment) | Half-Life Range (hours) | R² |
|---|---|---|
| 1 (0.5 mg/kg/day) | 34.53-41.74 | 0.95-0.99 |
| 2 (1.5 mg/kg/day) | 57.34-66.08 | 0.93-0.98 |
| 3 (5.0 mg/kg/day) | 57.51-72.09 | 0.88-0.98 |
| 4 (10.0 mg/kg/day) | 65.44-87.37 | 0.84-0.95 |

Half-life ranges were calculated using pK Solutions software (Summit Research Services, Montrose, Colo.) by varying the T₁ start point between 1 hour and 24 hours and the stop point being either 168 hours (Group 1) or 336 hours (Groups 2-4).

TABLE 7
Half-life of RG2077 (24 hour Start time)

| | Half-Life | | |
|---|---|---|---|
| Group | Range | | |
| (Treatment) | (hours) | Start Point-Final Point | R² |
| 1 (0.5 mg/kg/day) | 41.74-43.97 | 24-56 hours (168 hours) | 0.99-1.00 |
| 2 (1.5 mg/kg/day) | 66.08-71.13 | 24-96 hours (336 hours) | 0.98-1.00 |
| 3 (5.0 mg/kg/day) | 72.09-88.43 | 24-143 hours (336 hours) | 0.98-1.00 |
| 4 (10.0 | 87.37- | 24-143 hours (336 hours) | 0.95-1.00 |
| mg/kg/day) | 116.00 | | |

A two compartment pharmacokinetic model can be used to predict terminal half-life of RG2077. Schematically, it can be depicted as: 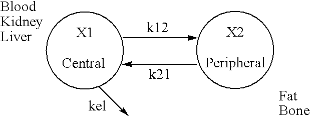
The differential equation for a drug in the central compartment following intravenous bolus administration is:

$$\frac{ⅆ X1}{ⅆ t} = - kel * X1 - k12 * X1 + k21 * X2$$

The kel*X1 term describes elimination of the drug from the central compartment, while the k12*×X1 and k21*X2 terms describe the distribution of the drug between the central and peripheral compartments. Integration of this equation (using Laplace transforms) leads to a biexponential equation for plasma concentration as a function of time.
Thus,
Cp=A*e^{-α*t}+B*e^{-β*t} with α>β.
The A, B, α, and β terms were derived from the microconstants during the integration process. They are functions of the microconstants k12, k21, kel and V1.
α+β=kel+k12+k21
α*β=kel*k21

$$\begin{matrix}\begin{matrix} α * β = \frac{( α + β ) + / - {[ {( α + β )}^{2} - 4 * α * β ]}^{1 / 2}}{2} \\ A = \frac{DOSE * ( α - k21 )}{{V}_{1} * ( α - β )} \end{matrix} \\ B = \frac{DOSE * ( k21 - β )}{{V}_{1} * ( α - β )}\end{matrix}$$

Using this two compartment pharmacokinetic model, all the different dose curves generate a good fit. Since it is possible to use this model to calculate the terminal half-life as t_{1/2}=0.693/β, it is possible to more accurately determine the terminal half-life of RG2077 for each dose group (Table 8).

TABLE 8
Half-life of RG2077 as determined by two compartment
pharmacokinetic model

| Dose group | | | | | Half-life |
|---|---|---|---|---|---|
| (mg/kg/day) | A | α | B | β | (hours) |
| 0.5 | 3.32 | 1.369 | 2.3 | 0.02495 | 27.8 |
| 1.5 | 13.7 | 0.514 | 4.4 | 0.01488 | 46.5 |
| 5 | 72.9 | 0.313 | 12 | 0.01089 | 63.6 |
| 10 | 111 | 0.246 | 19.9 | 0.01009 | 68.7 |

According to the model, the half-life of RG2077 was relatively similar in all of the volunteers and half-life increased slightly as the dose increased. Within each group, there was little variation in either the concentrations obtained at each time point or in the clearance rates between individuals.

Using a two-compartment pharmacokinetic model, the mean serum elimination half-life of RG2077 was calculated to be approximately 28 hours at 0.5 mg/kg, 46 hours at 1.5 mg/kg, 64 hours at 5 mg/kg, and 69 hours at 10 mg/kg. This suggests that, for doses less than 5 mg/kg, RG2077 was rapidly eliminated by a saturable mechanism.

Lymphocyte Population Analysis by Flow Cytometry

Blood samples were collected by PPD from volunteers in the RG2077-O2-04 study at Days 1 (pre-treatment), 4, 7, and either 27 or 28. Samples were stained using a commercially available cocktail of antibodies obtained from Beckman Coulter (Fullerton, Calif.). Samples were analyzed on a Beckman Coulter flow cytometer and cell numbers were obtained using fluorescent beads, included in each sample, to normalize values. Two samples were examined. The first sample was stained with antibodies that recognize the human antigens CD45, CD3, CD4 and CD8. The second sample contained antibodies against CD45, CD3, CD19, and CD56.

The populations discussed below were defined in the following manner:

| | | |
|---|---|---|
| T cells = | CD45⁺CD3⁺ | Sample 1 and 2 |
| CD4 T cells = | CD45⁺CD3⁺CD4⁺ | Sample 1 |
| CD8 T cells = | CD45⁺CD3⁺CD8⁺ | Sample 1 |
| B cells = | CD45⁺CD3⁻CD19⁺ | Sample 2 |
| NK Cells = | CD45⁺CD3⁻CD56⁺ | Sample 2 |

Absolute cell numbers were used to determine any changes in cell populations. The percent change at Day 4, 7, and 27/28 was calculated for each individual. These values were averaged to determine the mean change for each of the 5 groups (placebo, 0.5, 1.5, 5.0, and 10.0 mg/kg/day). This approach enabled the determination of the magnitude of change between any particular dosage and the placebo group.

Significant changes were observed in three instances. Two of these were an increase in the number of total T cells on Study Day 4 and Day 7 in the 10 mg/kg/day group (Day 4: p value=0.001; Day 7: p value=0.002). The third significant change was an increase in the number of CD4 T cells on Day 4 in the 10 mg/kg/day group (p value=0.034). Cell counts among the different groups were similar by day 28, suggesting that the earlier observed differences were transient.

These results suggest that there were few significant changes in the number of circulating lymphocytes. Also, the significant changes occurred at early time points following treatment and were resolved by the day 27/28 analysis. In addition, significant decreases were observed in the absolute cell count of any of the groups tested. Therefore, drug treatment did not appear to be toxic to circulating lymphocytes.

### Example 3

### Method for Treating an Autistic Patient with CTLA4-Ig

A 40 kg, seven-year-old male patient is diagnosed with autism according to the guidelines of the Diagnostic and Statistical Manual of Mental Disorders (DSM) (American Psychiatric Association, Washington, D.C., pages 66-71, 1994). The subject presents with a number of gastrointestinal symptoms indicative of DPI: GI cramping, chronic diarrhea, and gastroesophageal reflux (GER), and vomiting. A sample of the patient's blood is collected, and the level of IFN-γ is determined. The patient is treated with an injection of 200 mg CTLA4-Ig in the following formulation: 10 mg/l nL CTLA4-Ig, 150 mM NaCl, 20 mM potassium phosphate, 0.01% polysorbate 20. One day later, the subject's blood is drawn again, and the level of IFN-γ is determined. If the level of IFN-γ is diminished, a 200 mg dose of CTLA4-Ig is administered weekly for three additional weeks, and then monthly. The dosage can be adjusted as required to maintain a level of IFN-γ that corresponds to reduced symptoms.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating an individual exhibiting one or more symptoms of autism spectrum disorder (ASD), the method comprising administering an effective amount of a CTLA4 composition to the individual, wherein one or more symptoms of ASD are improved.

2. The method of claim 1, wherein the effective amount of CTLA4 in the CTLA4 composition comprises between about 0.1 mg/kg and 10 mg/kg.

3. The method of claim 1, wherein the CTLA4 composition is administered orally or transdermally.

4. The method of claim 1, wherein the CTLA4 composition is administered in a dose of 0.5, 1.5, 5.0, or 10.0 mg/kg.

5. The method of claim 1, wherein the CTLA4 composition comprises CTLA4-Ig.

6. A method for treating an individual exhibiting one or more symptoms of autistic syndrome disorder (ASD) and dietary protein intolerance (DPI), the method comprising administering an effective amount of a CTLA4 composition to the individual, wherein one or more symptoms of ASD or DPI are improved.

7. The method of claim 6, wherein the individual exhibits an elevated level of an inflammatory cytokine.

8. The method of claim 7, wherein the inflammatory cytokine is IFN-γ.

9. The method of claim 8, wherein the effective amount of the CTLA4 composition is sufficient to decrease a level of IFN-γ in the blood of the individual.

10. The method of claim 1, wherein the individual exhibits one or more gastrointestinal symptoms, and wherein one or more of the gastrointestinal symptoms is improved.

11. The method of claim 6, wherein the effective amount of CTLA4 in the CTLA4 composition comprises between about 0.1 mg/kg and 10 mg/kg.

12. The method of claim 6, wherein the CTLA4 composition is administered orally or transdermally.

13. The method of claim 6, wherein the CTLA4 composition is administered in a dose of 0.5, 1.5, 5.0, or 10.0 mg/kg.

14. The method of claim 6, wherein the CTLA4 composition comprises CTLA4-Ig.

15. A method for treating an individual exhibiting one or more symptoms of dietary protein intolerance (DPI), the method comprising administering an effective amount of a CTLA4 composition to the individual, and wherein one or more of the symptoms of DPI is improved.

16. The method of claim 15, wherein the effective amount of CTLA4 in the CTLA4 composition comprises between about 0.1 mg/kg and 10 mg/kg.

17. The method of claim 15, wherein the CTLA4 composition is administered orally or transdermally.

18. The method of claim 15, wherein the CTLA4 composition is administered in a dose of 0.5, 1.5, 5.0, or 10.0 mg/kg.

19. The method of claim 15, wherein the CTLA4 composition comprises CTLA4-Ig.
